(19) United States
(12) Patent Application Publication
(10) Pub. No.: US 2026/0295185 A1
(43) Pub. Date: Oct. 1, 2026

(54) TRACHEOSTOMY TUBES AND THEIR MANUFACTURE

(71) Applicant: ICU MEDICAL INTERNATIONAL LIMITED, Ashford (GB)

(72) Inventors: Timothy Bateman, Hythe (GB); Christopher John Woosnam, London (GB); Andrew Thomas Jeffrey, Romney Marsh (GB); Jonathan McNeill Flint, London (GB)

(73) Assignee: ICU MEDICAL INTERNATIONAL LIMITED, Ashford (GB)

(21) Appl. No.: 19/480,861

(22) PCT Filed: Apr. 4, 2024
(86) PCT No.: PCT/GB2024/000014

§ 371 Date: Nov. 3, 2025

(30) Foreign Application Priority Data
May 9, 2023 (GB) 2306819.0

(51) Int. Cl. A61M 16/04

(52) U.S. Cl. CPC A61M 16/0465; A61M 2207/00

(57) ABSTRACT

A tracheostomy tube has a shaft (10) with a flange (20) dividing the shaft into a patient end portion (22) for extending within the trachea and a machine end portion (23) for extending externally of the patient. The machine end portion (23) is reinforced by a plastics sleeve assembly (40) bonded to its outside. The sleeve assembly (40) includes a plastics sleeve member (41) having a shallow helical groove (43) around its outer surface. A plastic filament (42) is bonded into and around the groove to provide a helical reinforcing structure.

This invention relates to tracheostomy tubes of the kind having a shaft of a plastics material, a patient end portion of the shaft being adapted to extend into the patient's trachea and a machine end portion of the shaft being adapted to extend outwardly of the body.

Tracheal tubes are used to enable ventilation, respiration or spontaneous breathing of a patient. Endotracheal tubes are inserted via the mouth or nose so that one end locates in the trachea and the other end locates outside the patient. Tracheostomy tubes are inserted into the trachea via a surgically formed opening in the neck. Tracheostomy tubes can be inserted by different techniques, such as the surgical cut-down procedure carried out in an operating theatre or a cricothyroidotomy procedure, which may be carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. The tube is secured in position by means of a flange fixed towards the machine end of the tube and positioned to extend outwardly on opposite sides of the tube.

Tracheostomy tubes can be made of various materials and are usually of a bendable plastics material such as PVC, polyurethane or silicone. Where the tube is made of a very soft material, such as silicone, it increases the risk that it can be easily kinked and occluded by external pressure unless measures are taken to avoid this. Often, silicone tubes are reinforced by means of a stiff helical member extending along the tube, either along substantially their entire length, or along only a part of the length. Typically, the reinforcement member is a metal wire. Although metal wire reinforcements are ideal to give the degree of kink and crush resistance desired, they have the disadvantage of not being entirely compatible with MRI (magnetic resonance imaging) equipment or being only MRI conditional.

It has been proposed to use reinforcements made of non-ferromagnetic metals but there is reluctance to use even these materials, especially with higher resolution MRI equipment with field strengths of between 6 T and 10 T. MRI conditional materials may not cause a danger to the patient, but they can distort the magnetic field and cause image artefacts.

It has also been proposed to use stiffer plastics filaments, such as of nylon or aramid, as the helical reinforcement of silicone tubes. These can provide some degree of reinforcement although not as much as metals. These plastics have a further disadvantage that they are often not as heat resistant as silicone so they can be prone to damage by the high temperatures met during autoclave treatment.

Some tracheostomy tubes have a machine end portion of the shaft extending outwardly beyond the point where the tube enters the body. A silicone paediatric tracheostomy tube sold by ICU Medical under the Bivona® FlexTend™ trade mark (Bivona is a Registered Trade Mark of ICU Medical) has such a machine end portion extending outwardly beyond the supporting neck flange. The machine end portion of this tube is reinforced with a helical metal wire and is terminated by a connector by which connection is made to the tube. Because the machine end portion extends freely outside the body and is connected to breathing tubing it would be particularly prone to kinking if not reinforced.

Other examples of reinforced tracheal tubes are described in, for example, GB2552250, GB933307, U.S. Pat. No. 5,906,036, WO2010/089523, EP1078645, WO2015075412, WO08083286, U.S. Pat. Nos. 6,148,818, 5,546,936, 5,429,127, EP0950424, EP2644221, U.S. Pat. Nos. 5,628,787, 4,737,153, US2012/0118294, U.S. Pat. Nos. 4,990,143, 5,181,509, US2007083132, U.S. Pat. Nos. 8,783,254, 6,130,406, US2015/101611, U.S. Pat. No. 6,017,335 and DE867144. WO2019/180398 describes a tracheostomy tube with a portion of its shaft extending externally of the patient that is reinforced by a small-bore plastics tube wrapped helically around the external portion of the shaft and bonded to it. This can effectively reinforce the external portion and avoids the use of a ferromagnetic material. The problem, however, with such a tube is that it is labour-intensive to make, so the tube is relatively expensive.

It is an object of the present invention to provide an alternative reinforced tracheostomy tube and its method of manufacture.

According to one aspect of the present invention there is provided a tracheostomy tube of the above-specified kind, characterised in that the machine end portion is reinforced along at least a part of its length by a separately formed plastics sleeve assembly incorporating a helical formation, and that the sleeve assembly is secured with the outer surface of the machine end portion.

The helical formation is preferably provided by an elongate member of a non-ferromagnetic material, such as a plastics material, for example a silicone. The sleeve assembly preferably includes a sleeve member to which the elongate member is secured, and both the sleeve member and the elongate member are preferably of plastics material with the material of the elongate member being harder than that of the sleeve member.

The helical formation may be a solid filament or a hollow tube.

The sleeve assembly preferably includes a helical groove along a part at least of its length, the helical formation being bonded in the groove.

According to another aspect of the present invention there is provided a method of manufacturing a tracheostomy tube including the steps of providing a tubular, plastics shaft having a patient end portion adapted to extend into a patient's trachea and a machine end portion adapted to extend outwardly of the trachea, providing a separately formed plastics sleeve assembly incorporating a helical formation, and securing the sleeve assembly with the outer surface of the machine end portion of the shaft.

The plastics sleeve assembly preferably has a helical groove extending along a part at least of its length, the method including the step of applying an adhesive or solvent to the groove and wrapping an elongate member along the groove.

According to a further aspect of the present invention there is provided a tracheostomy tube made by a method according to the above other aspect of the present invention.

A paediatric tracheostomy tube and its method of manufacture both according to the present invention will now be described, by way of example, with reference to the accompanying drawings, in which:

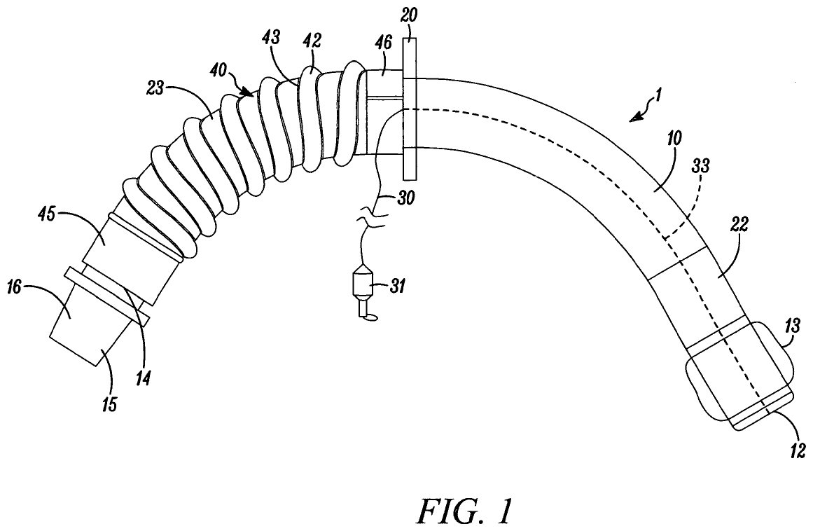

FIG. 1 is a side elevation view of the tracheostomy tube; and

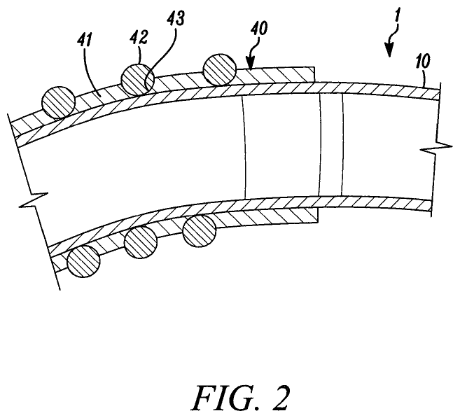

FIG. 2 is an enlarged cross-sectional, side elevation view of a part of the machine end portion of the tube of FIG. 1.

The tracheostomy tube 1 has a curved shaft 10 of circular section formed from a flexible silicone material having a durometer (Shore A) between 60 and 70. The dimensions of the tube are selected to be suitable for use in paediatric or neonatal patients. The shaft 10 has a patient end 12 adapted to be located within the trachea of the patient and has a conventional sealing cuff 13 towards its patient end. Tubes with different dimensions could be used in adult patients.

The machine end 14 of the shaft 10 is adapted, during use, to be located externally of the tracheostomy opening formed in the patient's neck. The machine end 14 of the shaft 10 is bonded with a hub or connector 15 having a conventional 15 mm male tapered outer surface 16. The connector 15 is adapted to make a removable push fit in a conventional 15 mm female connector (not shown) at one end of a breathing tube extending to a ventilator or anaesthetic machine. Alternatively, the machine end of the tube 1 could be left open to atmosphere when the patient is breathing spontaneously. The tracheostomy tube 1 also includes a radially extending support flange 20 adapted to lie against the skin surface of the neck on either side of the tracheostomy stoma. The flange 20 may be moulded integrally with the shaft 10 about midway along its length or it could be a separate component. The flange 20 has openings (not shown) at opposite ends for attachment to a neck strap (not shown) used to support the tube with the patient's neck. The flange 20 divides the shaft 10 into a patient end portion 22, adapted to extend through the tracheostomy and locate in the trachea, and a machine end portion 23 that extends rearwardly, outwardly of the tracheostomy and extends freely from the patient. This machine end portion 23 provides a flexible connection of the connector 15 with the patient end portion 22 so that the tracheostomy is isolated to some extent from the connector. This enables connection to and disconnection from the connector 15, or repositioning of the connector, to be made with less force being transmitted to the tracheostomy tissue, thereby reducing trauma and discomfort.

It will be appreciated that the tube could have a different size or shape and different features according to the application. The patient end portion 22 and machine end portion 23 of the shaft 10 are preferably (as shown) different portions of a common shaft but could be different components joined end-to-end.

The sealing cuff 13 is inflated and deflated by means of an inflation line 30 terminated at one end by a conventional combined inflation indicator and an MRI-compatible valved connector 31. The other end of the inflation line 30 is connected, in the region of the flange 20, to an inflation lumen 33 extending along one side of the patient end portion 22 of the shaft 10.

The silicone material from which the shaft 10 is made has various advantages. It is highly flexible, very compatible with patient tissue, and is resistant to the high temperatures used during autoclave heat treatment. The flexible nature of the material, however, means that the machine end portion 23, in particular, would be prone to kinking unless steps were taken to prevent this. Present silicone tubes with this configuration, therefore, usually have their machine end portion reinforced by a helical metal wire. Such an arrangement prevents this portion kinking without substantially reducing its flexibility. The metal wire, however, means that the tube is not compatible with MRI equipment. Even where the reinforcing metal wire is non-ferromagnetic it may still cause distortion of the MRI image, especially at higher field strengths.

In the arrangement of the present invention the machine end portion 23 of the shaft 10 is reinforced by a separate sleeve assembly 40 extending along and bonded to the outside of the machine end portion. The reinforcing sleeve assembly 40 includes a sleeve member 41 and some form of helical formation 42, which together increase the strength of the machine end portion against compression and kinking but still enable the machine end portion 23 to flex freely. In the arrangement illustrated the helical formation takes the form of a filament 42 of circular section wound around the sleeve member 41 and bonded in a helical groove 43 formed around and along the outside of the major part of the sleeve member. The diameter of the filament 42 is approximately twice the depth of the groove 43 so that the filament projects above the outer surface of the sleeve member 41. The external projection of the filament 42 does not cause a clinical problem because this part of the tube 1 does not extend within the body. The projecting filament 42 could help improve the grip on the tube 1 when this needs to be removed from the patient. The filament 42 and sleeve member 41 could be made of the same plastics material and may be of the same material as the shaft 10, such as silicone. The filament 42 could be of a harder material than the sleeve member 41, or a different, harder grade of the same material such as a silicone elastomer with a durometer (Shore A) of about 80. The filament 42 and groove 43 are shown as not extending to the very end of the sleeve member 41 but are spaced from the ends by short unreinforced collars 45 and 46. The unreinforced collar 45 at the machine end 14 of the tube 1 gives it slightly more resilience so as to enable the patient end of the connector 15 to be inserted more easily into the machine end of the shaft 10. The helically wound filament 42 provides a reinforcing structure that resists lateral compression, crushing and kinking of the machine end portion 23 but still allows this portion to flex and bend.

The tube 1 is manufactured by forming the shaft 10 in any conventional way, such as by extrusion, moulding, dipping or the like, and by separately preforming the sleeve assembly 40. The sleeve assembly 40 is slipped onto the shaft 10 from the machine end 14 using an adhesive or a solvent such as room temperature vulcanising (RTV) liquid silicone applied between the shaft and the sleeve assembly to act as a lubricant until the sleeve assembly is in the desired position. It is then left in position until the solvent or adhesive has cured. Heat or UV radiation may be used to promote curing. The sealing cuff 13, inflation line 30, 33 and connector 15 can be assembled in the usual way.

The sleeve assembly 40 is formed as a separate component from the shaft 10 by moulding the sleeve member 41 with its external helical groove 43. The groove 43 provides an accurate guide into which an adhesive can be applied and enables the manufacturing assemblers readily to lay the filament 42 along the groove. This ensures that assembly can be carried out rapidly, accurately and reproducibly.

The pitch of the groove 43 and helical formation 42 need not be constant along the length of the reinforced section but could vary so as to vary the reinforcement, so that it is greatest where needed. The helical formation need not be provided by a solid filament but could be a hollow, small-bore tube. The filament or tube need not be circular in section but could be oval or rectilinear, such as square or rectangular. In such a case the groove would have a corresponding profile. The helical formation could be formed by overmoulding a thin layer of a material stiffer than the underlying sleeve so that this fills the groove to form a helical winding. The sleeve could be moulded in one piece and integrally with the neck flange 20 of the tube 1 and, or alternatively, with the machine end connector 15 so that these can be assembled on the shaft 10 of the tube at the same time. This reduces the risk of any of these items detaching from the shaft during insertion or use.

The reinforcing sleeve 40 enables the machine end portion 23 to flex freely with a low risk of kinking. Because the reinforcement 40 is made of the same material (although of a different grade) as the main part of the shaft 10 it bonds well with the shaft. It is also resistant to autoclave temperatures, thereby enabling the tube 1 to be autoclaved after use and reused. This helps reduce the cost of maintaining a patient with a tracheostomy tube and also reduces the cost and environmental damage caused by disposal of clinical waste. The reinforcement 40 enables the tube 1 to be used safely in MRI environments, even those with high field strengths.

The invention is particularly advantageous in paediatric size tubes because the small diameter shafts in such tubes makes them more prone to kinking but is not confined to paediatric sizes.

1-11. (canceled)

12. A tracheostomy tube having a shaft of a plastics material, a patient end portion of the shaft being adapted to extend into the a patient's trachea and a machine end portion of the shaft having a length and an outer surface being adapted to extend outwardly of the patient's body, characterised in that the machine end portion is reinforced along at least a part of the length by a separately formed plastics sleeve assembly incorporating a helical formation, and that the sleeve assembly is secured with the outer surface of the machine end portion.

13. The tracheostomy tube according to claim 12, characterised in that the helical formation is provided by an elongate member of a non-ferromagnetic material.

14. The tracheostomy tube according to claim 13, characterised in that the elongate member (42) is of a plastics material.

15. The tracheostomy tube according to claim 14, characterised in that the elongate member is of a silicone.

16. A tracheostomy tube according to claim 13, characterised in that the sleeve assembly includes a sleeve member to which the elongate member is secured, and that both the sleeve member and the elongate member are of plastics material where the material of the elongate member is harder than that of the sleeve member.

17. The tracheostomy tube according to claim 12, characterised in that the helical formation is a solid filament.

18. The tracheostomy tube according to claim 12, characterised in that the helical formation is a hollow tube.

19. The tracheostomy tube according to claim 12, characterised in that the sleeve assembly includes a helical groove along a part at least of a length of the sleeve assembly, and that the helical formation is bonded in the groove.

20. A method of manufacturing a tracheostomy tube comprising:
providing a tubular, plastics shaft having a patient end portion adapted to extend into a patient's trachea and a machine end portion having an outer surface adapted to extend outwardly of the patient's trachea;
providing a separately formed plastics sleeve assembly incorporating a helical formation; and
securing the sleeve assembly with the outer surface of the machine end portion of the shaft.

21. The method according to claim 20, characterised in that the plastics sleeve assembly has a length and a helical groove extending around an outside of the sleeve assembly along at least a part of the length, the method further comprising:
applying an adhesive or solvent to the groove and wrapping the helical formation along the groove.

22. A tracheostomy tube made by the method according to claim 20.
